(19) United States
(12) Patent Application Publication
(10) Pub. No.: US 2025/0367331 A1
(43) Pub. Date: Dec. 4, 2025

(54) MEDICAL HYDROGEL CONTAINING ULTRASONICALLY-IMAGEABLE BUBBLE MICROSPHERES AND PREPARATION METHOD THEREOF

(71) Applicant: SHANGHAI REUNION MEDICAL CO., LTD., Jiading District, Shanghai (CN)

(72) Inventors: Yali WANG, Jiading District, Shanghai (CN); Churong WANG, Jiading District, Shanghai (CN); Zhanpeng ZHANG, Jiading District, Shanghai (CN); Zhen PAN, Jiading District, Shanghai (CN)

(73) Assignee: SHANGHAI REUNION MEDICAL CO., LTD., Jiading District, Shanghai (CN)

(21) Appl. No.: 19/143,518

(22) PCT Filed: Jul. 5, 2024
(86) PCT No.: PCT/CN2024/103865

§ 371 Date: Jun. 26, 2025

(30) Foreign Application Priority Data
Aug. 3, 2023 (CN) 202310973760.X

(51) Int. Cl. A61K 49/22; A61K 9/50; A61K 9/51

(52) U.S. Cl. CPC A61K 49/223; A61K 9/5031; A61K 9/5052; A61K 9/5192; A61K 49/226

(57) ABSTRACT

Provided are a medical hydrogel containing ultrasonically-imageable bubble microspheres and a preparation method thereof. The medical hydrogel containing the ultrasonically-imageable bubble microspheres is formed by in-situ crosslinking of a polyethylene glycol precursor solution and a poly-amino crosslinker solution containing bubble microspheres, the polyethylene glycol precursor solution consisting of a multi-arm polyethylene glycol derivative and a buffer solution A.

### CROSS-REFERENCE TO RELATED APPLICATION

The present application is a national stage application of International Patent Application No. PCT/CN2024/103865, filed on Jul. 5, 2024, which claims priority to Chinese Patent application No. CN 202310973760.X, filed with the China National Intellectual Property Administration on Aug. 3, 2023 and entitled “MEDICAL HYDROGEL CONTAINING ULTRASONICALLY-IMAGEABLE BUBBLE MICROSPHERES AND PREPARATION METHOD THEREFOR”. The disclosure of the two applications is incorporated herein by reference in its entirety.

### TECHNICAL FIELD

The present disclosure relates to the technical field of pharmaceutical formulations, and particularly relates to a medical hydrogel containing ultrasonically-imageable bubble microspheres and a preparation method thereof.

### BACKGROUND

Hydrogel is a soft material containing substantial amounts of moisture obtained by crosslinking a hydrophilic polymer. The hydrogel boasts excellent physical and chemical properties and biological characteristics, such as strong conformability, biodegradability, and good biocompatibility. Consequently, hydrogel products have received a lot of attention in the biomedical field, including use in drug delivery, spacing and protection, and implantation and intervention like vascular embolization.

However, after implantation in the body, hydrogels could not be imaged using conventional ultrasound imaging techniques. This limitation makes it difficult for researchers to conveniently and effectively monitor location, shape, and degradation status of the implanted hydrogels in vivo. Although considerable research has focused on enhancing imaging properties of the hydrogels, there are currently very few effective methods available. Most approaches rely on introducing materials of different densities than water to achieve ultrasound imageability, for example, introducing water-insoluble imaging particles into the hydrogel. Chinese patent (Publication No. CN 116077744 A) discloses an absorbable self-imaging hydrogel, and a preparation method and use thereof. Imaging function of the hydrogel is derived from inorganic nanoparticles generated by in-situ bonding of anions and salt ions in components of a buffer solution. However, this method presents several issues, such as poor imaging performance, a delay in imaging after implantation, and potential changes in properties of the hydrogel. In addition, although pre-incorporating imaging particles could provide the hydrogels with enhanced imaging effect, these imaging particles are typically dense and prone to rapid sedimentation, leading to unstable performance; moreover, the most contentious issue is that water-insoluble imaging particles may remain in the body for an extended period after the hydrogel has degraded and could potentially migrate, which raises concerns about risks of foreign body reactions and thrombosis.

### SUMMARY

In order to solve the problems mentioned above, the present disclosure provides a medical hydrogel containing ultrasonically-imageable bubble microspheres and a preparation method thereof. While not affecting gelling performance of a hydrogel, the provided medical hydrogel could be clearly ultrasonically imaged, thus realizing real-time observation of the hydrogel in human bodies. In addition, the medical hydrogel is completely degradable without residues.

An aspect of the present disclosure provides a medical hydrogel containing ultrasonically-imageable bubble microspheres, where the medical hydrogel containing the ultrasonically-imageable bubble microspheres is formed by in-situ crosslinking of a polyethylene glycol precursor solution and a poly-amino crosslinker solution containing bubble microspheres, the polyethylene glycol precursor solution including (or consisting of) a multi-arm polyethylene glycol derivative and a buffer solution A.

In some embodiments, the multi-arm polyethylene glycol derivative is at least one selected from the group consisting of a three-arm polyethylene glycol derivative, a four-arm polyethylene glycol derivative, a six-arm polyethylene glycol derivative, and an eight-arm polyethylene glycol derivative.

In some embodiments, the multi-arm polyethylene glycol derivative is an aldehyde group-terminated multi-arm polyethylene glycol derivative, where an aldehyde group in the aldehyde group-terminated multi-arm polyethylene glycol derivative is linked to a multi-arm polyethylene glycol via one linkage selected from the group consisting of an ether linkage, an amide linkage, a urethane linkage, an imine linkage, and a urea linkage.

In some embodiments, the multi-arm polyethylene glycol derivative has a number average molecular weight of 10 kDa to 20 kDa.

In some embodiments, the multi-arm polyethylene glycol derivative is a four-arm polyethylene glycol terminated with a benzaldehyde group linked via an amide linkage, and has a number average molecular weight of 10 kDa.

In some embodiments, a mass fraction of the multi-arm polyethylene glycol derivative in the polyethylene glycol precursor solution is in a range of 10% to 24%, and preferably 10% to 20%.

In some embodiments, the poly-amino crosslinker solution containing the bubble microspheres is prepared by raw materials including at least a poly-amino compound serum albumin, and a buffer solution B.

In some embodiments, the poly-amino compound includes (or consists of) at least one selected from the group consisting of trilysine, polyethyleneimine and polylysine, preferably a composition of the polyethyleneimine and the polylysine.

In some embodiments, a mass fraction of the poly-amino compound in the poly-amino crosslinker solution containing the bubble microspheres is in a range of 9% to 17%, and preferably 9.128% to 16.13%. In some embodiments, a concentration ratio of the polyethyleneimine to the polylysine is in a range of 4-7:5-11.

The medical hydrogel provided by the present disclosure is prepared by crosslinking four-arm polyethylene glycol terminated with a benzaldehyde group linked via an amide linkage, which has a number average molecular weight of 10 kDa, with polyethyleneimine and polylysine, followed by curing. With a mass fraction of a multi-arm polyethylene glycol derivative in the polyethylene glycol precursor solution further controlled to be 10% to 24%, the polyethylene glycol precursor solution is then mixed with a poly-amino crosslinker solution containing bubble microspheres in which a mass fraction of a poly-amino compound is 9% to 17%, followed by forming a gel within a relatively short period of gelation time. The gelation time is ensured to be less than 10 seconds on average, and meanwhile, a cured hydrogel has good stability and a low swelling ratio. If the mass fraction of the poly-amino compound is lower, it will cause the gelation time to be too long to satisfy the requirements of actual applications.

In some embodiments, a mass fraction of the serum albumin in the poly-amino crosslinker solution containing the bubble microspheres is in a range of 10% to 30%, and preferably 10% to 20%. In some embodiments, the serum albumin is bovine serum albumin.

Given limitations in the prior art, in some embodiments of the present disclosure, a poly-amino crosslinker solution containing bovine serum albumin bubble microspheres is prepared by adding bovine serum albumin to a crosslinking system, and after mixing with a polyethylene glycol precursor solution, two components are crosslinked in situ and cured to form an ultrasonically-imageable medical hydrogel, the hydrogel could be imaged clearly under ultrasound due to a low density of gas. Further, the inventors discovered during research that with a mass fraction of serum albumin in a poly-amino crosslinker solution containing bubble microspheres controlled to be 10% to 20%, it not only does not affect gelling performance of the hydrogel and allows the hydrogel to be completely degradable without residues, but also contributes to the stability of the hydrogel. The inventors analyzed that possible reasons could be: amino groups contained in a bovine serum albumin (BSA) shell of the bubble microspheres could chemically bond with a multi-arm polyethylene glycol derivative, such that the bubble microspheres could be stably loaded into the hydrogel through chemical bonding over a long period and gradually degraded and released as the hydrogel degrades, thus ensuring stability of ultrasound imaging of the hydrogel and synchronization with degradation of the hydrogel, and facilitating real-time observation of the hydrogel in human bodies. However, an excessive addition of the BSA could accelerate a degradation rate, and insufficient addition of the BSA could affect imaging performance.

In some embodiments, the buffer solution A and the buffer solution B are independently a phosphate buffer solution with a pH of 4 to 10 or a borax buffer solution with a pH of 4 to 10; and preferably, the buffer solution A is the phosphate buffer solution with the pH of 5.6; and the buffer solution B is the borax buffer solution with the pH of 9.2.

Another aspect of the present disclosure provides a method for preparing the medical hydrogel containing ultrasonically-imageable bubble microspheres described above, including at least the following steps:
(1) dissolving the multi-arm polyethylene glycol derivative in the buffer solution A to prepare the polyethylene glycol precursor solution; and
(2) injecting the polyethylene glycol precursor solution and the poly-amino crosslinker solution containing the bubble microspheres at a volume ratio in a mixed manner by using a dual-chamber syringe to obtain the medical hydrogel containing the ultrasonically-imageable bubble microspheres.

In some embodiments, the poly-amino crosslinker solution containing the bubble microspheres is prepared by a process as follows: dissolving the poly-amino compound in the buffer solution B to prepare a base solution of the poly-amino crosslinker solution; dissolving serum albumin in the base solution of the poly-amino crosslinker solution while controlling a temperature of a resulting system; after complete dissolution, subjecting a resulting solution to ultrasonic treatment and nitrogen purging simultaneously; upon completion, immediately placing a treated solution into a constant-temperature water bath tank and conducting low-temperature treatment; and then taking out and returning to 20° C. to 30° C. to obtain the poly-amino crosslinker solution containing the bubble microspheres.

In some embodiments, a method for preparing the medical hydrogel containing the ultrasonically-imageable bubble microspheres specifically includes the following steps:
S1: dissolving the multi-arm polyethylene glycol derivative in the phosphate buffer solution with the pH of 5.6 to prepare the polyethylene glycol precursor solution;
S2: dissolving the poly-amino compound in the borax buffer solution with the pH of 9.2 to prepare the base solution of the poly-amino crosslinker solution;
S3: dissolving the BSA in the base solution of the poly-amino crosslinker solution while controlling a temperature of a resulting system; after complete dissolution, subjecting the resulting solution to the ultrasonic treatment and the nitrogen purging simultaneously; upon completion, immediately placing the treated solution into the constant-temperature water bath tank and conducting the low-temperature treatment; and then taking out and returning to 20° C. to 30° C. to obtain the poly-amino crosslinker solution containing the bovine serum albumin bubble microspheres;
S4: injecting the polyethylene glycol precursor solution and the poly-amino crosslinker solution containing the bubble microspheres at the volume ratio in the mixed manner by using a dual-chamber syringe to obtain the medical hydrogel containing the ultrasonically-imageable bubble microspheres.

In some embodiments, in step S3, the temperature is controlled to be 55° C. to 65° C.

In some embodiments, in step S3, the ultrasonic treatment and the nitrogen purging each are conducted for 10 seconds to 30 seconds and repeated 1 time to 5 times.

In some embodiments, in step S3, the low-temperature treatment is conducted at a temperature of −5° C. to 0° C. for 40 minutes to 60 minutes.

In some embodiments, the volume ratio of the polyethylene glycol precursor solution to the poly-amino crosslinker solution containing the bubble microspheres is 1:1.

The present disclosure inventively incorporates serum albumin (e.g., bovine serum albumin) bubble microspheres into a two-component gel system using a specific process, addressing various issues present in the existing imaging systems that use water-insoluble imaging particles, such that a provided ultrasonically-imageable hydrogel material has potential to offer new materials for the medical and tissue engineering fields, and could be applied in areas such as implantable medical devices, a permanent embolization, and artificial tissue scaffolds, enabling clear ultrasound imaging observation during and after an implantation process.

A third aspect of the present disclosure provides use of the medical hydrogel containing the ultrasonically-imageable bubble microspheres in implantable medical devices, a permanent embolization, and artificial tissue scaffold.

### Beneficial Effects

1. The present disclosure provides a medical hydrogel containing ultrasonically-imageable bubble microspheres. While not affecting gelling performance of a hydrogel, the provided medical hydrogel could be clearly ultrasonically imaged, thus enabling real-time observation of the hydrogel in human bodies. In addition, the medical hydrogel is completely degradable without residues.
2. In the present disclosure, with a mass fraction of a multi-arm polyethylene glycol derivative in a polyethylene glycol precursor solution controlled to be 10% to 24%, the polyethylene glycol precursor solution is then mixed with a poly-amino crosslinker solution containing bubble microspheres in which a mass fraction of a poly-amino compound is 9% to 17%, followed by forming a gel within a relatively short period of gelation time. The gelation time is ensured to be less than 10 seconds on average, and meanwhile, a cured hydrogel has good stability and a low swelling ratio.
3. In the present disclosure, a poly-amino crosslinker solution containing bovine serum albumin bubble microspheres is prepared by adding bovine serum albumin to a crosslinking system; after mixing with a polyethylene glycol precursor solution, two components are crosslinked in situ and cured to form an ultrasonically-imageable medical hydrogel, the hydrogel could be imaged clearly under ultrasound due to a low density of gas.
4. In the present disclosure, with a mass fraction of serum albumin in the poly-amino crosslinker solution controlled to be 10% to 30%, it not only does not affect gelling performance of the hydrogel and allows the hydrogel to be completely degradable without residues, but also contributes to the stability of the hydrogel.
5. The present disclosure inventively incorporates the bovine serum albumin bubble microspheres into a two-component gel system using a specific process, addressing various issues present in the existing imaging systems that use water-insoluble imaging particles, such that a provided ultrasonically-imageable hydrogel material has potential to offer new materials for the medical and tissue engineering fields, and could be applied in areas such as implantable medical devices, a permanent embolization, and artificial tissue scaffolds, enabling clear ultrasound imaging observation during and after an implantation process.

### BRIEF DESCRIPTION OF THE DRAWINGS

FIG. 1 shows a comparison graph of the swelling ratios over time for Example 3, along with Examples 1, 2 and 4, and Comparative example 2;

FIG. 2 shows a comparison graph of the swelling ratios over time for Example 3, along with Examples 5 and 6, and Comparative example 2;

FIG. 3 shows a comparison graph of the swelling ratios over time for Example 3, along with Examples 9 and 10, and Comparative example 2;

FIG. 4A shows a morphological characterization image of the bovine serum albumin bubble microspheres under an optical microscope at 10× magnifications for Example 3;

FIG. 4B shows a morphological characterization image of the bovine serum albumin bubble microspheres under an optical microscope at 20× magnifications for Example 3;

FIG. 4C shows a morphological characterization image of the bovine serum albumin bubble microspheres under an optical microscope at 10× magnifications for Example 9;

FIG. 4D shows a morphological characterization image of the bovine serum albumin bubble microspheres under an optical microscope at 20× magnifications for Example 9;

FIG. 5A shows an ultrasound imaging effect diagram of Comparative example 2, with implanted materials marked by a circle;

FIG. 5B shows an ultrasound imaging effect diagram of Example 3, with implanted materials marked by a circle; and

FIG. 5C shows an ultrasound imaging effect diagram of Example 9, with implanted materials marked by a circle.

### DETAILED DESCRIPTION OF THE EMBODIMENTS

### Example 1

An aspect of Example 1 of the present disclosure provided a medical hydrogel containing ultrasonically-imageable bubble microspheres, where the medical hydrogel containing the ultrasonically-imageable bubble microspheres was formed by in-situ crosslinking of a polyethylene glycol precursor solution and a poly-amino crosslinker solution containing bubble microspheres. The polyethylene glycol precursor solution consisted of a multi-arm polyethylene glycol derivative and a buffer solution A.

The multi-arm polyethylene glycol derivative was four-arm polyethylene glycol terminated with a benzaldehyde group linked via an amide linkage, with a number average molecular weight of 10 kDa (purchased from JenKem Technology Co., Ltd. Beijing, China).

A mass fraction of the multi-arm polyethylene glycol derivative in the polyethylene glycol precursor solution was 10%.

Raw materials for preparing the poly-amino crosslinker solution containing the bubble microspheres consisted of a poly-amino compound, serum albumin, and a buffer solution B.

A mass fraction of the poly-amino compound in the poly-amino crosslinker solution containing the bubble microspheres was 16.13%, where the poly-amino compound was a combination of polylysine and polyethyleneimine, and a concentration ratio of the polyethyleneimine to the polylysine was 6.078:10.052.

A mass fraction of the serum albumin in the poly-amino crosslinker solution containing the bubble microspheres was 20%. The serum albumin was bovine serum albumin.

The buffer solution A is a phosphate buffer solution with a pH of 5.6; and the buffer solution B was a borax buffer solution with a pH of 9.2.

Another aspect of Example 1 of the present disclosure provided a method for preparing the medical hydrogel containing the ultrasonically-imageable bubble microspheres, the method was specifically preformed according to the following steps.
S1: A polyethylene glycol derivative was dissolved in 1 mL of the buffer solution A and to prepare the polyethylene glycol precursor solution.
S2: The poly-amino compound was dissolved in 1 mL of the buffer solution B to prepare a base solution of the poly-amino crosslinker solution.
S3: The bovine serum albumin was dissolved in the base solution of the poly-amino crosslinker solution while controlling a temperature of a resulting system; after complete dissolution, a resulting solution was subjected to ultrasonic treatment and nitrogen purging simultaneously; upon completion, a treated solution was immediately placed into a constant-temperature water bath tank and subjected to low-temperature treatment; and then a resulting material was taken out and returned to 25° C. to obtain the poly-amino crosslinker solution containing bovine serum albumin bubble microspheres.
S4: The polyethylene glycol precursor solution and the poly-amino crosslinker solution were injected in a mixed manner at a volume ratio by using a dual-chamber syringe to obtain the medical hydrogel containing the ultrasonically-imageable bubble microspheres.

In step S3, the temperature was controlled to be 60° C. In step S3, the ultrasonic treatment and the nitrogen purging each were conducted 1 time for 15 seconds.

In S3, the low-temperature treatment was conducted at a temperature of −5° C. for 45 minutes.

A volume ratio of the polyethylene glycol precursor solution to the poly-amino crosslinker solution containing the bovine serum albumin bubble microspheres was 1:1.

### Example 2

Example 2 of the present disclosure provided a medical hydrogel containing ultrasonically-imageable bubble microspheres and a preparation method thereof. The specific embodiments of Example 2 were the same as Example 1, except that a mass fraction of a multi-arm polyethylene glycol derivative in a polyethylene glycol precursor solution was 13%.

### Example 3

Example 3 of the present disclosure provided a medical hydrogel containing ultrasonically-imageable bubble microspheres and a preparation method thereof. The specific embodiments of Example 3 were the same as Example 1, except that a mass fraction of a multi-arm polyethylene glycol derivative in a polyethylene glycol precursor solution was 20%.

### Example 4

Example 4 of the present disclosure provided a medical hydrogel containing ultrasonically-imageable bubble microspheres and a preparation method thereof. The specific embodiments of Example 4 were the same as Example 1, except that a mass fraction of a multi-arm polyethylene glycol derivative in a polyethylene glycol precursor solution was 24%.

### Example 5

Example 5 of the present disclosure provided a medical hydrogel containing ultrasonically-imageable bubble microspheres and a preparation method thereof. The specific embodiments of Example 5 were the same as Example 1, except that a mass fraction of a poly-amino compound in a poly-amino crosslinker solution containing bubble microspheres was 13.153%, where the poly-amino compound was a combination of polylysine and polyethyleneimine, and a concentration ratio of the polyethyleneimine to the polylysine was 5.539:7.614.

### Example 6

Example 6 of the present disclosure provided a medical hydrogel containing ultrasonically-imageable bubble microspheres and a preparation method thereof. The specific embodiments of Example 6 were the same as Example 1, except that a mass fraction of a poly-amino compound in a poly-amino crosslinker solution containing bubble microspheres was 9.128%, where the poly-amino compound was a combination of polylysine and polyethyleneimine, and a concentration ratio of the polyethyleneimine to the polylysine was 4.052:5.076.

### Example 7

Example 7 of the present disclosure provided a medical hydrogel containing ultrasonically-imageable bubble microspheres and a preparation method thereof. The specific embodiments of Example 7 were the same as Example 1, except that a mass fraction of a poly-amino compound in a poly-amino crosslinker solution containing bubble microspheres was 8.065%, where the poly-amino compound was a combination of polylysine and polyethyleneimine, and a concentration ratio of the polyethyleneimine to the polylysine was 3.039:5.026.

### Example 8

Example 8 of the present disclosure provided a medical hydrogel containing ultrasonically-imageable bubble microspheres and a preparation method thereof. The specific embodiments of Example 8 were the same as Example 1, except that a mass fraction of a poly-amino compound in a poly-amino crosslinker solution containing bubble microspheres was 6.515%, where the poly-amino compound was a combination of polylysine and polyethyleneimine, and a concentration ratio of the polyethyleneimine to the polylysine was 2.076:4.439.

### Example 9

Example 9 of the present disclosure provided a medical hydrogel containing ultrasonically-imageable bubble microspheres and a preparation method thereof. The specific embodiments of Example 9 were the same as Example 1, except that a mass fraction of serum albumin in a poly-amino crosslinker solution containing bubble microspheres was 10%.

### Example 10

Example 10 of the present disclosure provided a medical hydrogel containing ultrasonically-imageable bubble microspheres and a preparation method thereof. The specific embodiments of Example 10 were the same as Example 1, except that a mass fraction of serum albumin in a poly-amino crosslinker solution containing bubble microspheres was 30%.

### Comparative Example 1

Comparative example 1 of the present disclosure provided a medical hydrogel containing ultrasonically-imageable bubble microspheres and a preparation method thereof. The specific embodiments of Comparative example I were the same as Example 1, except that a mass fraction of serum albumin in a poly-amino crosslinker solution containing bubble microspheres was 40%.

### Comparative Example 2

Comparative example 2 of the present disclosure provided a medical hydrogel containing ultrasonically-imageable bubble microspheres and a preparation method thereof. The specific embodiments of Comparative example 2 were the same as Example 1, except that a mass fraction of serum albumin in a poly-amino crosslinker solution containing bubble microspheres was of 0%.

### Performance Test Methods

1. Gelation time: 0.6 mL of the polyethylene glycol precursor solution and 0.6 mL of the poly-amino crosslinker solution containing the bubble microspheres in each of the examples and in Comparative Example 2 were taken into 2.5 mL of vials. The 2.5 mL of vials were placed in a 37±0.5° C. water bath and left to stand for 5 min. 200 μL of the polyethylene glycol precursor solution was transferred into a 2.5 mL vial using a pipette; then after changing a pipette tip, 200 μL of the poly-amino crosslinker solution containing the bubble microspheres was transferred to the vial charged with the polyethylene glycol precursor solution. The vial was manually shaken at a frequency of 3 times/second for 3 seconds, and then the vial was repeatedly tilted at a frequency of 1 time/second for observation until the liquid did not flow. A timing commenced as soon as the polyethylene glycol precursor solution was added and was stopped when the liquid did not flow. The recorded duration was the gelation time. Three groups of samples were measured in parallel and results were averaged. The results were recorded in Table 1.

TABLE 1
Gelation time of polyethylene glycol precursor solutions
in each example and Comparative example 2

| Example | Gelation time (second) | | | Mean |
|---|---|---|---|---|
| Example 1 | 9.12 | 9.78 | 9.83 | 9.58 |
| Example 2 | 9.06 | 8.78 | 8.94 | 8.93 |
| Example 3 | 6.72 | 7.6 | 7.03 | 7.12 |
| Example 4 | 6.5 | 6.75 | 6.32 | 6.52 |
| Example 5 | 9.16 | 8.43 | 8.56 | 8.72 |
| Example 6 | 9.25 | 10.03 | 9 | 9.43 |
| Example 7 | 30.6 | 32.59 | 27.59 | 30.26 |
| Example 8 | 77.5 | 66.57 | 86.05 | 76.71 |
| Example 9 | 4.97 | 5.62 | 5.16 | 5.25 |
| Example 10 | 18.27 | 18.37 | 18.91 | 18.52 |
| Comparative example 1 | Not to gel | | | — |
| Comparative example 2 | 3.82 | 4.34 | 3.94 | 4.03 |

2. Degradation performance: The hydrogels provided in all examples except Example 7, Example 8 and Comparative example 1, and in Comparative Example 2 were subjected to an accelerated aging experiment (a simulated in vitro degradation experiment, where the hydrogels were incubated in a constant-temperature oven at 60° C.). Complete degradation time of the hydrogels was recorded. Results are shown in Table 2. Data in Table 2 demonstrate that the complete degradation time of the hydrogel products provided varies from 7 days to 14 days (with 60° C., 7 days approximately equivalent to 37° C., 34 days; 60° C., 9 days approximately equivalent to 37° C., 44 days; 60° C., 11 days approximately equivalent to 37° C., 54 days; and 60° C., 14 days approximately equivalent to 37° C., 69 days), thus the hydrogel products could meet needs of various clinical uses for different degradation times.

TABLE 2
Complete degradation time of hydrogels provided in
Examples 1-6 and 9-10 and Comparative example 2

| Hydrogel product | Complete degradation time (60° C.) (Day) |
|---|---|
| Comparative example 2 | 7 |
| Example 1 | 9 |
| Example 2 | 9 |
| Example 3 | 11 |
| Example 4 | 14 |
| Example 5 | 14 |
| Example 6 | 7 |
| Example 9 | 14 |
| Example 10 | 9 |

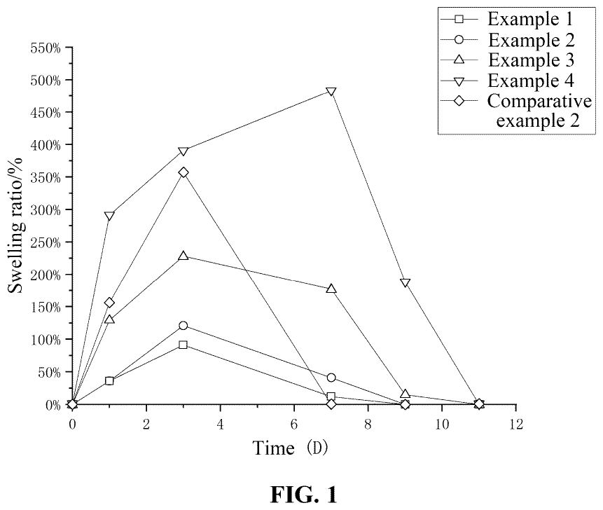

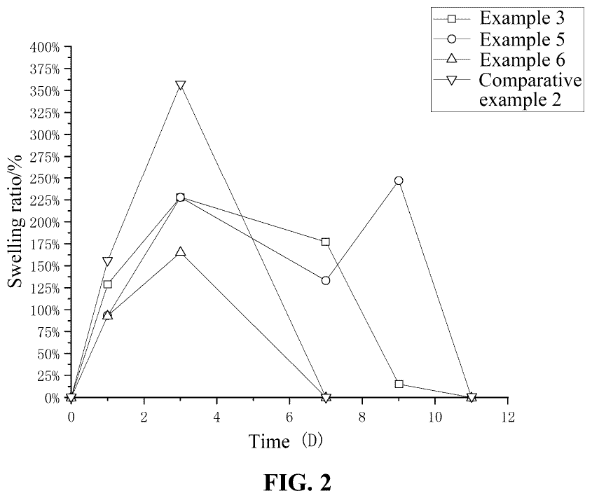

3. Swelling ratio of hydrogels: Based on the degradation performance data of the hydrogels provided by the examples and Comparative example 2, the swelling ratios of the hydrogels were calculated. Results are shown in FIG. 1, FIG. 2, FIG. 3, and Table 3. The comparison indicates that the hydrogel products provided by Example 3 and Example 9 exhibit better hydrogel stability and lower hydrogel swelling ratios.

TABLE 3
Swelling ratio of hydrogels provided in Examples
1-6 and 9-10 and Comparative example 2

| | Time (day) | | | | | | |
|---|---|---|---|---|---|---|---|
| Swelling ratio (%) | 0 | 1 | 3 | 7 | 9 | 11 | 14 |
| Example 1 | 0 | 36 | 91 | 12 | 0 | 0 | — |
| Example 2 | 0 | 36 | 121 | 41 | 0 | 0 | — |
| Example 3 | 0 | 129 | 228 | 177 | 15 | 0 | — |
| Example 4 | 0 | 291 | 391 | 483 | 188 | 0 | — |
| Example 5 | 0 | 93 | 228 | 133 | 247 | 0 | — |
| Example 6 | 0 | 93 | 165 | 0 | 0 | 0 | — |
| Example 9 | 0 | 147 | 225 | 223 | 98 | — | 0 |
| Example 10 | 0 | 185 | 397 | 371 | 0 | 0 | — |
| Comparative example 2 | 0 | 156 | 357 | 0 | 0 | 0 | — |

Note:
(“—” means not tested)

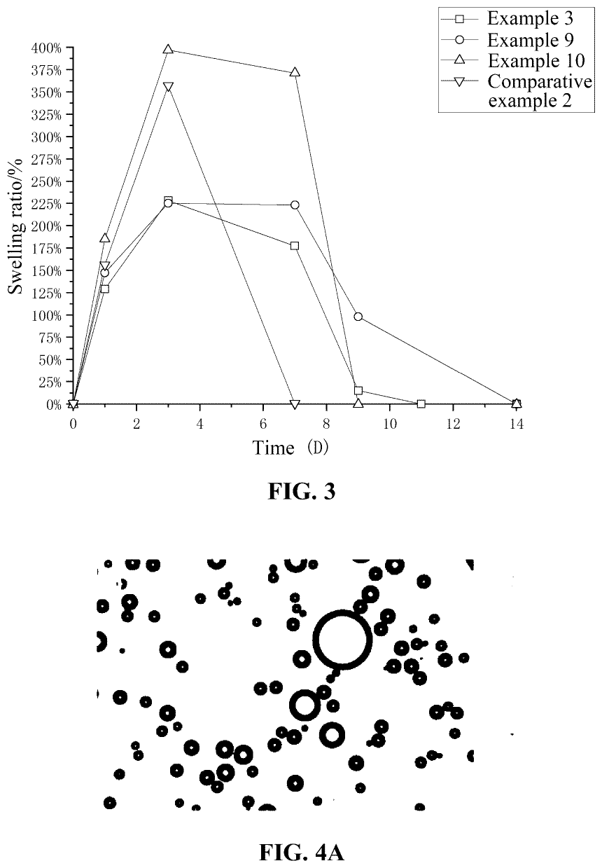

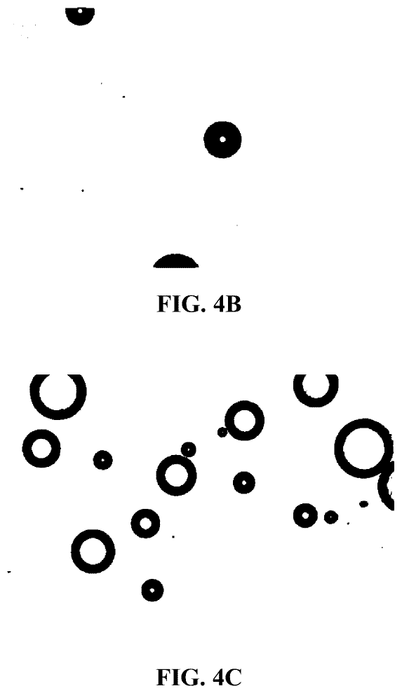

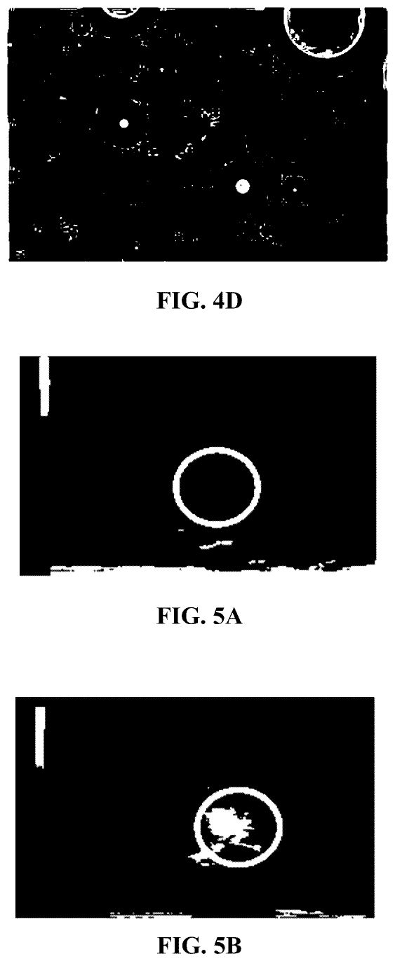

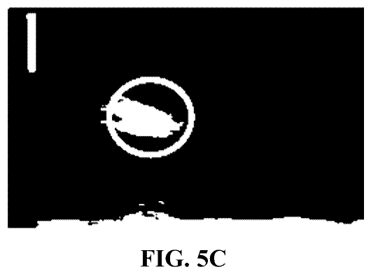

4. The hydrogels provided by Example 3 and Example 9 were placed under an optical microscope to observe a shape of bovine serum albumin (BSA) bubble microspheres. It was found that the number of the BSA bubble microspheres formed in Example 3 was significantly higher than that in Example 9. Results are shown in FIG. 4A to FIG. 4D.
5. The hydrogels provided by Example 3 (FIG. 5B), Example 9 (FIG. 5C), and Comparative example 2 (FIG. 5A) were implanted subcutaneously in rats for ultrasound imaging. Comparative example 2 (FIG. 5A) could not be imaged under ultrasound, while Example 3 (FIG. 5B) demonstrates superior ultrasound imaging effects compared to Example 9 (FIG. 5C). Ultrasound imaging results are shown in FIG. 5A to FIG. 5C.

1. A medical hydrogel containing ultrasonically-imageable bubble microspheres, wherein the medical hydrogel containing the ultrasonically-imageable bubble microspheres comprises bubble microspheres and a crosslinked product at surfaces of the bubble microspheres;
and the crosslinked product is prepared by crosslinking a multi-arm polyethylene glycol derivative with a poly-amino crosslinker.

2. A medical hydrogel containing ultrasonically-imageable bubble microspheres, wherein the medical hydrogel containing the ultrasonically-imageable bubble microspheres is formed by in-situ crosslinking of a polyethylene glycol precursor solution and a poly-amino crosslinker solution containing bubble microspheres, the polyethylene glycol precursor solution comprising a multi-arm polyethylene glycol derivative and a buffer solution A; and
the buffer solution A has a pH of 4 to 10.

3. The medical hydrogel containing the ultrasonically-imageable bubble microspheres of claim 2, wherein the poly-amino crosslinker solution containing the bubble microspheres is prepared by raw materials comprising at least a poly-amino compound, serum albumin, and a buffer solution B; and the buffer solution B has a pH of 4 to 10.

4. The medical hydrogel containing the ultrasonically-imageable bubble microspheres of claim 1, wherein the multi-arm polyethylene glycol derivative is at least one selected from the group consisting of a three-arm polyethylene glycol derivative, a four-arm polyethylene glycol derivative, a six-arm polyethylene glycol derivative, and an eight-arm polyethylene glycol derivative.

5. The medical hydrogel containing the ultrasonically-imageable bubble microspheres of claim 1, wherein the multi-arm polyethylene glycol derivative is an aldehyde group-terminated multi-arm polyethylene glycol derivative, wherein an aldehyde group in the aldehyde group-terminated multi-arm polyethylene glycol derivative is linked to a multi-arm polyethylene glycol via one linkage selected from the group consisting of an ether linkage, an amide linkage, a urethane linkage, an imine linkage, and a urea linkage.

6. The medical hydrogel containing the ultrasonically-imageable bubble microspheres of claim 2, wherein a mass fraction of the multi-arm polyethylene glycol derivative in the polyethylene glycol precursor solution is in a range of 10% to 24%.

7. The medical hydrogel containing the ultrasonically-imageable bubble microspheres of claim 3, wherein the poly-amino compound comprises at least one selected from the group consisting of trilysine, polyethyleneimine and polylysine.

8. The medical hydrogel containing the ultrasonically-imageable bubble microspheres of claim 3, wherein a mass fraction of the poly-amino compound in the poly-amino crosslinker solution containing the bubble microspheres is in a range of 9% to 17%.

9. The medical hydrogel containing the ultrasonically-imageable bubble microspheres of claim 3, wherein a mass fraction of the serum albumin in the poly-amino crosslinker solution containing the bubble microspheres is in a range 10% to 30%.

10. The medical hydrogel containing the ultrasonically-imageable bubble microspheres of claim 3, wherein the buffer solution A and the buffer solution B are independently a phosphate buffer solution with a pH of 4 to 10 or a borax buffer solution with a pH of 4 to 10.

11. A method for preparing the medical hydrogel containing the ultrasonically-imageable bubble microspheres of claim 1, comprising at least the following steps:
(1) dissolving the multi-arm polyethylene glycol derivative in a buffer solution A to obtain a polyethylene glycol precursor solution; and
(2) injecting the polyethylene glycol precursor solution and a poly-amino crosslinker solution containing bubble microspheres at a volume ratio in a mixed manner by using a dual-chamber syringe to obtain the medical hydrogel containing the ultrasonically-imageable bubble microspheres.

12. The method of claim 11, wherein the poly-amino crosslinker solution containing the bubble microspheres is prepared by a process as follows: dissolving a poly-amino compound in a buffer solution B to prepare a base solution of the poly-amino crosslinker solution; dissolving serum albumin in the base solution of the poly-amino crosslinker solution while controlling a temperature of a resulting system to be 55° C. to 65° C.; after complete dissolution, subjecting a resulting solution to ultrasonic treatment and nitrogen purging simultaneously; upon completion, placing a treated solution into a constant-temperature water bath tank and conducting low-temperature treatment; and then taking out and returning to 20° C. to 30° C. to obtain the poly-amino crosslinker solution containing the bubble microspheres.

13. The method of claim 12, wherein the ultrasonic treatment and the nitrogen purging are conducted for 10 seconds to 30 seconds and repeated 1 time to 5 times.

14. The method of claim 12, wherein the low-temperature treatment is conducted at a temperature of −5° C. to 0° C. for 40 minutes to 60 minutes.

15. A method of using the medical hydrogel containing the ultrasonically-imageable bubble microspheres of claim 1 in a medical device.

16. The method of claim 15, wherein the medical device comprises one selected from the group consisting of a permanent embolization material and an artificial tissue scaffold.

17. The medical hydrogel containing the ultrasonically-imageable bubble microspheres of claim 2, wherein the multi-arm polyethylene glycol derivative is at least one selected from the group consisting of a three-arm polyethylene glycol derivative, a four-arm polyethylene glycol derivative, a six-arm polyethylene glycol derivative, and an eight-arm polyethylene glycol derivative

18. The medical hydrogel containing the ultrasonically-imageable bubble microspheres of claim 2, wherein the multi-arm polyethylene glycol derivative is an aldehyde group-terminated multi-arm polyethylene glycol derivative, wherein an aldehyde group in the aldehyde group-terminated multi-arm polyethylene glycol derivative is linked to a multi-arm polyethylene glycol via one linkage selected from the group consisting of an ether linkage, an amide linkage, a urethane linkage, an imine linkage, and a urea linkage.

19. The medical hydrogel containing the ultrasonically-imageable bubble microspheres of claim 7, wherein a mass fraction of the poly-amino compound in the poly-amino crosslinker solution containing the bubble microspheres is in a range of 9% to 17%.

20. A method for preparing the medical hydrogel containing the ultrasonically-imageable bubble microspheres of claim 2, comprising at least the following steps;
(1) dissolving the multi-arm polyethylene glycol derivative in the buffer solution A to obtain the polyethylene glycol precursor solution; and
(2) injecting the polyethylene glycol precursor solution and the poly-amino crosslinker solution containing the bubble microspheres at a volume ratio in a mixed manner by using a dual-chamber syringe to obtain the medical hydrogel containing the ultrasonically-imageable bubble microspheres
